(19) United States
(12) Patent Application Publication
(10) Pub. No.: US 2026/0232925 A1
(43) Pub. Date: Aug. 13, 2026

(54) A METHOD FOR DETECTING A STATE OF STALL IN A ROTATING STEPPING MOTOR

(71) Applicant: SHL Medical AG, Zug (CH)

(72) Inventor: Ola Hallström, Nacka Strand (SE)

(21) Appl. No.: 19/155,829

(22) PCT Filed: Jan. 26, 2024
(86) PCT No.: PCT/EP2024/051849

§ 371 Date: Aug. 12, 2025

Related U.S. Application Data
(60) Provisional application No. 63/449,091, filed on Mar. 1, 2023.

(51) Int. Cl. A61M 5/50; A61M 5/20; H02P 8/36

(52) U.S. Cl. CPC A61M 5/5086; H02P 8/36; A61M 2005/2026; A61M 2205/103; A61M 2205/3365; A61M 2205/50

(57) ABSTRACT

The present disclosure provides a method for detecting a state of stall in a rotating stepping motor, the method comprising the steps of: (i) measuring the back EMF voltage of the stepping motor when the stepping motor is rotating, (ii) calculating a first spread of the back EMF voltage, wherein the first spread of the back EMF is defined by a difference be a highest back EMF value and a lowest back EMF value in a first fluctuation of the back EMF voltage when the stepping motor is rotating. (iii) calculating a second spread of the back EMF voltage, wherein the second spread of the back EMF is defined by a difference be a highest back EMF value and a lowest back EMF value in a second fluctuation of the back EMF voltage when the stepping motor is rotating, (iv) comparing the second spread to the first spread, and (v) determining the stepping motor is stalled when a difference between the second spread and the first spread is higher than a first predetermined value.

### TECHNICAL FIELD

The present disclosure generally relates to a method for detecting a state of stall in a rotating stepping motor, and particularly to a method comprising the steps of measuring back EMF value.

### BACKGROUND

Medicament delivery devices such as pen-type manual injectors or auto-injectors are generally known for self-administration of a medicament by patients without formal medical training. For example, patients suffering from diabetes may require repeated injections of insulin, or patients may require regular injections of other types of medicaments, such as growth hormones.

It is an advantage when the medicament delivery devices for self-administration comprise automatic functions so that even if users are without professional training or are in an emergency situation, the users can easily and properly use the medicament delivery devices.

Sensor-less stall/step loss detection in a stepping motor-based systems are demanded in various systems as the stepping motor is frequently used for precisely controlling the rotation angle and speed. In particular, when the stepping motor in a medicament delivery device to drive a plunger rod, a position of the plunger rod can be determined based on the calculation of the rotation of the stepping motor. As the position of the plunger rod usually directed to the information such as amount of delivered dose/residual amount of dose, the accuracy of the detection of the plunger rod position is crucial for the dose accuracy. Thus, detecting whether the stepping motor is stalled can increase the detection of the position of the plunger rod in a stepping-motor based, sensor-less system and also prevents the motor from damage. The stall situation sometimes happen due to multiple reasons, e.g., a pulse signal has a short cycle or when the load of the stepping motor is large.

Some common ways of the stall detection extract back Electro Motive Force (EMF) from a non-excited coil and use a threshold of the back EMF to detect stall or step-loss. The threshold is either dynamically derived based on rotation speed or statically fixed or calibrated for a given system. However, the threshold used must be chosen very generically and with a safety margin that takes various complicating factors into account, such as temperature of the motor windings, battery condition and motor individual properties and tolerances. Thus, the safety margin to prevent stall might have to be so big that the available max torque is severely reduced.

Even though many of the devices on the market, as well as the ones described above, have their respective advantages, there is still room for improvement.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a method for detecting a state of stall in a rotating stepping motor, the method comprising the following steps with the following order: measuring the back EMF voltage value of the rotating stepping motor; calculating a first spread of the back EMF voltage value, wherein the first spread of the back EMF voltage value is defined by a difference between a highest back EMF voltage value and a lowest back EMF value in a first fluctuation of the back EMF voltage value when the stepping motor is rotating; calculating a second spread of the back EMF voltage value, wherein the second spread of the back EMF voltage value is defined by a difference between a highest back EMF voltage value and a lowest back EMF voltage value in a second fluctuation of the back EMF voltage value when the stepping motor is rotating; comparing the second spread to the first spread; and determining that the stepping motor is stalled when a difference between the second spread and the first spread is higher than a first predetermined value.

In a preferred example, the method for detecting a state of stall in a rotating stepping motor of a medicament delivery device.

Preferably, according to another embodiment, the step of measuring the back EMF voltage value of the rotating stepping motor further comprises the following steps with the following order: recording a back EMF voltage value; and starting to calculate the first spread and the second spread only when the recorded back EMF voltage value is lower than a second predetermined value.

Preferably, according to another embodiment, the second predetermined value is defined by the following steps with the following order: obtaining the rotation speed of the stepping motor; and determining the second predetermined value by searching data from a first predetermined table with the obtained rotation speed of the stepping motor.

Preferably, according to another embodiment, the step of obtaining the rotation speed of the stepping motor comprises the step of: measuring the rotation speed of the stepping motor.

Alternatively, according to another embodiment, the step of obtaining the rotation speed of the stepping motor comprises the step of: receiving signal containing a predetermined rotation speed of the stepping motor.

Preferably, according to another embodiment, the method further comprising the following steps with the following order; wherein the following steps with the following order can be performed before or after the any steps according to any of the preceding claims, measuring the back EMF voltage value of the rotating stepping motor; comparing the measured value to a third predetermined value; and determining a value of load of the stepping motor.

Preferably, according to another embodiment, before the step of determining the value of load of the stepping motor, the method comprises the steps of: obtaining the rotation speed of the stepping motor; and determining the third predetermined value by searching data from a second predetermined table with the obtained rotation speed of the stepping motor.

Preferably, according to another embodiment, the above-mentioned method is configured to be performed by a processor of a drive unit of a medicament delivery device. The drive unit comprises a stepping motor.

Preferably, according to another embodiment, the drive unit comprises a stepping motor, a plunger rod operably connected to the stepping motor, and the above-mentioned processor, the processor being electrically connected to the stepping motor, wherein the plunger rod is configured to be driven by the stepping motor when the stepping motor is rotating.

Preferably, according to another embodiment, the processor is configured to pause, slow, or restore the rotation of the stepping motor when the processor determines the stepping motor is stalled.

Preferably, according to another embodiment, the processor is configured to determine a position of the plunger rod based on detecting the rotation of the stepping motor.

Preferably, according to another embodiment, the drive unit comprises a housing configured to receive a cassette that is releasably attached to the housing; and wherein the plunger rod is configured to be moved into the medicament container within the cassette.

Preferably, according to another embodiment, the drive unit comprises a switch movable between an inactive position where the processor and the motor are both inactive and an active position wherein the processor is activated.

Preferably, according to another embodiment, the switch is configured to be moved from the inactive position to the active position when the cassette is attached to the housing.

Preferably, according to another embodiment, the plunger rod is connected to the stepping motor via a lead screw.

Preferably, according to another embodiment, the stepping motor is rotatable around the lead screw within the housing. The stepping motor is engaged with the lead screw via a threaded engagement. A switch is arranged within the housing. Upon activation of the switch, the rotation of the stepping motor around the lead screw is configured to move the lead screw proximally so that a stopper of a medicament container in the cassette is moved by the lead screw.

Preferably, according to another embodiment, the stepping motor is the stepping motor.

Preferably, according to another embodiment, the motor comprises a rotor and a channel extending through the rotor. The lead screw is partially positioned within the channel.

Preferably, according to another embodiment, the channel comprises a thread extending around the channel. The thread is engaged with the thread of the lead screw.

Alternatively, according to another embodiment, the drive unit comprises a connector operably connected to the rotor such that the connector is configured to be rotated by the rotor.

Preferably, according to another embodiment, a channel extends through the connector and is aligned with the channel of the rotor along the longitudinal axis.

Preferably, according to another embodiment, the channel of the connector comprises a thread extending around the channel of the connector. The thread is engaged with the thread of the lead screw.

Preferably, according to another embodiment, the connector is attached to the rotor.

Preferably, according to another embodiment, the connector and the rotor are made of different materials.

Preferably, according to another embodiment, the connector is made of plastic.

Preferably, according to another embodiment, the rotor is made of metal.

Preferably, according to another embodiment, the longitudinal axis is the central axis of the motor. Preferably, according to another embodiment, an engagement formed between the plunger rod and the inner wall of the housing is a ribs-ribs engagement or a ribs-grooves engagement.

Preferably, according to another embodiment, an engagement formed between the lead screw and the inner wall of the housing is a ribs-ribs engagement or a ribs-grooves engagement.

Preferably, according to another embodiment, the distal end of the lead screw comprises a polygonal cross-section perpendicular to the longitudinal axis and the inner wall of the housing comprises a polygonal cross-section perpendicular to the longitudinal axis.

Preferably, according to another embodiment, the stepping motor is configured to reciprocate the movement of the lead screw.

Preferably, according to another embodiment, the drive unit comprises a second switch accommodated within the housing.

Preferably, according to another embodiment, the second switch is aligned with the distal end of the lead screw in the direction of the longitudinal axis.

Preferably, according to another embodiment, the second switch is positioned distally away from the distal end of the lead screw.

Preferably, according to another embodiment, the second switch is configured to be switched by the distal end of the lead screw when the lead screw is moved distally relative to the stepping motor such that when the second switch is switched the motor is stopped.

Preferably, according to another embodiment, the second switch is a non-contact switch.

Alternatively, according to another embodiment, the second switch is configured to be switched by being pushed in the distal direction relative to the housing by the distal end of the lead screw. Preferably, according to another embodiment, the second switch comprises a flipping arm.

Preferably, according to another embodiment, the second switch is configured to be switched when the flipping arm is pivoted by the distal end of the lead screw.

Preferably, according to another embodiment, the housing extends along the longitudinal axis between a proximal end and a distal end.

Preferably, according to another embodiment, the housing comprises a fixture at a proximal portion of the housing.

Preferably, according to another embodiment, the fixture is configured to be releasably attached to a counter fixture of the cassette of the medicament delivery device.

Preferably, according to another embodiment, the fixture and the counter fixture form a bayonet connection or a thread connection.

Preferably, according to another embodiment, the switch is a non-contact switch.

Preferably, according to another embodiment, the drive unit comprises a communication unit.

Preferably, according to another embodiment, the communication unit is configured to receive information from the cassette.

Preferably, according to another embodiment, the communication unit is configured to be activated when the switch is in the active position.

Preferably, According to Another Embodiment, the Switch Is a Non-contact Switch.

Alternatively, according to another embodiment, the switch is operably movable by a component of the cassette of the medicament delivery device when the cassette is attached to the housing between an active position where the switch is activated and an inactive position where the third switch is deactivated.

Preferably, according to another embodiment, the medicament container of the medicament delivery device is a syringe, a cartridge or a collapsible bag.

Preferably, according to another embodiment, the medicament container of the medicament delivery device is made of glass material or plastic material.

Preferably, according to another embodiment, the medicament container of the medicament delivery device is accommodated within the cassette.

Preferably, according to another embodiment, the medicament delivery device is an injection device, an inhalation device, or a medical sprayer.

Preferably, according to another embodiment, the injection device is configured to perform a subcutaneous injection, intramuscular injection, or intravenous injection.

Preferably, according to another embodiment, the medicament delivery device is an auto-injector. Preferably, according to another embodiment, the medicament delivery member is an injection needle or a spray nozzle.

Preferably, according to another embodiment, the medicament delivery member is accommodated in the cassette.

Preferably, according to another embodiment, the cassette is configured to be fully received within the housing of the drive unit.

Preferably, according to another embodiment, the cassette is configured to be partially received within the housing of the drive unit.

Preferably, according to another embodiment, the cassette comprises an information carrier.

Preferably, according to another embodiment, the information tag is one of an RFID chip, NFC chip, barcode, and QR code.

Preferably, according to another embodiment, the information carrier is attached to an outer surface of a body of the cassette.

Preferably, according to another embodiment, the information carrier is embedded in the body.

Preferably, according to another embodiment, the information carrier is attached to the medicament container.

Preferably, according to another embodiment, the information carrier is a flexible sheet.

Preferably, according to another embodiment, the body having a counter fixture releasably attached to the fixture of the housing of the drive unit and a delivery member cover.

Preferably, according to another embodiment, the body extends along an axis between a proximal end and a distal end.

Preferably, according to another embodiment, a delivery member cover is telescopically relative to a proximal end of the body between an extended position where the delivery member cover protrudes from the proximal end of the body in the direction of the axis and a retracted position.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary drugs that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary drugs that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1 /PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary drugs that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC 500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VelP, OFF, GTX, CAV, AD, MAID, AIM, VAC-IE, ADOC, or PE.

Exemplary drugs that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a medicament delivery device with a drive unit of the invention;

FIG. 2 schematically shows a perspective view of components of the drive unit of FIG. 1;

FIG. 3 shows an example of the measured of the back EMF value of a rotating stepping motor; and

FIG. 4 shows a flow chart of the method of the invention.

### DETAILED DESCRIPTION

FIG. 4 shows a method for detecting a state of stall in a rotating stepping motor. Preferably, the method for detecting a state of stall in a rotating stepping motor of a medicament delivery device. The method comprises the steps of firstly measuring the back EMF value of the rotating stepping motor 401; secondly, calculating a fluctuation amplitude (a difference between a highest back EMF value and a lowest back EMF value) of the back EMF value fluctuation (is called ‘spread’ in the description), in other words, calculating a first spread of the back EMF value 402; thirdly, calculating a second spread of the back EMF value 403, the second spread occurs after the first spread occurs; afterward, comparing the second spread to the first spread 404; and finally, determining the stepping motor is stalled 406 when a difference between the second spread and the first spread is higher than a first predetermined value 405. If the difference between the second spread and the first spread is not higher than a first predetermined value, the method restarts again from the calculating a first spread of the back EMF value 402. It should be noted that the first spread happens previously than the second spread. The period between the first spread and the second spread is dependent on the desgin.

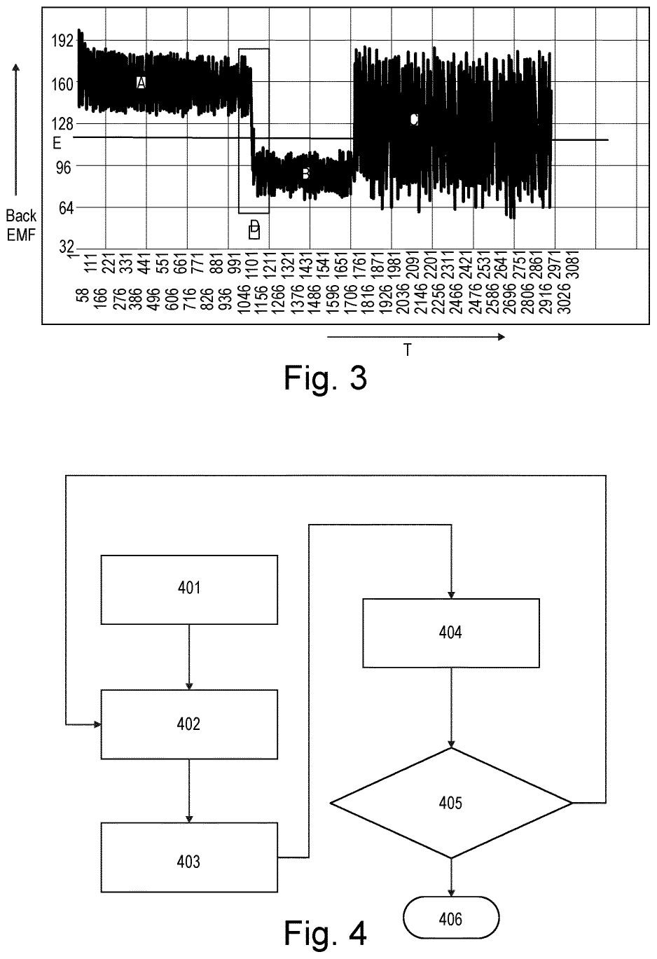

As the back EMF value will fluctuate (oscillation), the method is configured to dynamically measure and compare the fluctuation amplitude of the back EMF value fluctuation (is called ‘spread’ in the description). Instead of simply using a pre-set threshold, the spread of the back EMF value is analysed and compared to previously collected data. As the back EMF value oscillation will look different for varying loads as well as when stalling, as shown in FIG. 3, the stall of the rotating stepping motor can be detected. Furthermore, the back EMF value oscillation will always be present in a rotating motor, even for very low loads. Therefore, the stepping motor can use its full available motor power instead of being stopped earlier due to a generic set safety margin to prevent stall. Furthermore, as the fluctuation of the back EMF value can be measured in a short time period, the stall can be detected very early once it happens before damage the motor.

FIG. 3 shows one example of a measurement of the back EMF value/time T of a rotating stepping motor. Section A of the measured back EMF value is measured when the stepping motor starts to rotate and there is no load connected to the motor. Section B of the measured back EMF value is measured when the stepping motor is rotating and is connected to 60 newton (N) load connected. Section C of the measured back EMF value is measured when the stepping motor is rotating, and the motor is stalled. As the spread of the back EMF value is greater when the motor is stalled (in comparison with the motor is not stalled), comparing the spread that is happened later (the second spread) to the previous measured spread (the first spread), the motor stall can be detected.

In a preferred example, the steps of measuring the back EMF value of the rotating stepping motor further comprises the steps of: recording a back EMF value; and starting to calculate the first and the second spreads only when the recorded back EMF value is lower than a second predetermined value E.

As can be seen from FIG. 3, when the rotating stepping motor is connected to the load, a great spread will be created (section D in FIG. 3). To prevent false detection, it is preferred that before measuring the spread, the back EMF value will be record and compared to the second predetermined value, to make sure that the stepping motor is connected to load (in other words, the motor is used). As can be seen from FIG. 3, the back EMF value is lower when the motor is connected to the load. Thus, the second predetermined value E can be defined. In other words, a detection based on a result of the comparison between the first spread and the second spread and a result of the comparison between the detected back EMF value and the second predetermined value can increase the accuracy of detecting the state of stall in a rotating stepping motor.

Furthermore, in another preferred example, the second predetermined value is defined by the steps of obtaining the rotation speed of the stepping motor; and determining the second predetermined value by searching data from a first predetermined table with the obtained rotation speed of the stepping motor.

As back EMF value will be affected also by multiple factors, such as the rotating speed and/or the max spread levels, the second predetermined value E can be dynamically defined by looked up in a first predetermined table for the particular speed.

The rotation speed of the stepping motor can be obtained by either measuring the rotation speed or receiving signal containing a predetermined rotation speed of the stepping motor, e.g., input by a user or read RFID tag on the motor.

Some other measures can be done by measuring the back EMF value. For example, measuring the back EMF value of the stepping motor when the stepping motor is rotating to determine a value of load of the stepping motor by comparing the measured value to a third predetermined value. The third predetermined value can be determined by searching data from a second predetermined table with the obtained rotation speed of the stepping motor.

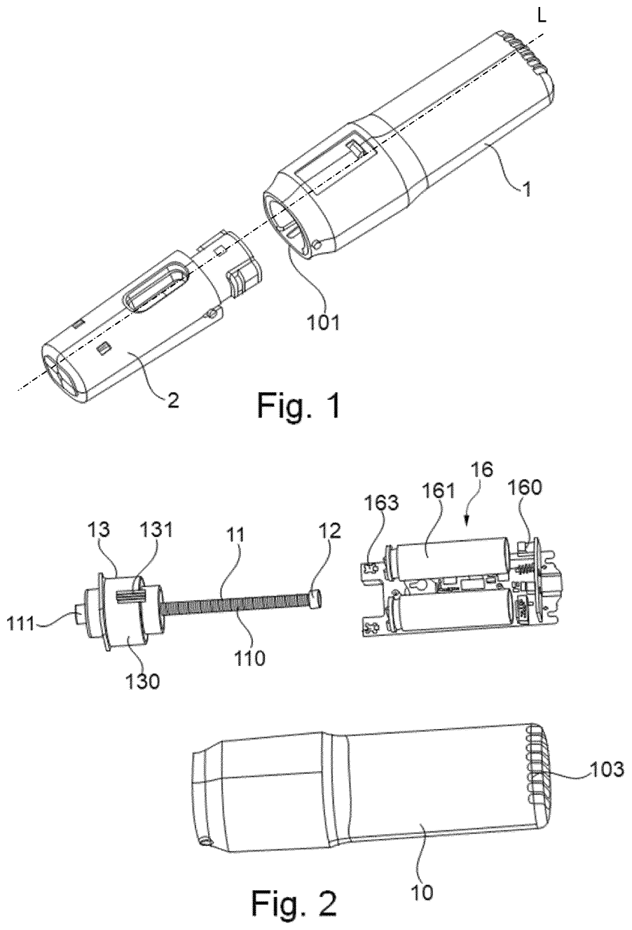

FIGS. 1-2 illustrate a medicament delivery device comprises a drive unit 1. The drive unit comprises a stepping motor 13 and a processor electrically connected to the stepping motor 13, as shown in FIG. 2, and is configured to perform the above-mentioned method. In a preferred example, the drive unit 1 is reusable and is configured to releasably attached to a cassette 2.

Preferably, the cassette 2 is disposable.

The housing 10 is configured to accommodate a portion of the cassette 2. In one example, the housing 10 comprises a fixture 101 configured to be releasably engaged with the counter fixture 202 of the cassette 2. Preferably, the fixture 101 and the counter fixture 202 form a bayonet engagement, as shown in FIG. 1. In this example, the cassette 2 is partially received within the housing 10. Alternatively, the cassette can be fully received within the housing of the drive unit. In this example, the fixture and the counter fixture are not necessary, as the cassette can be made with a shape that is matched with a receiving chamber of the housing of the drive unit.

The housing 10 comprises an electronic section 16. The electronic section 16 comprises a PCB 160 comprising the processor, e.g., MCU or CPU, a battery 161, a switch 163 and optionally at least one of a second switch, a communication unit, a vibration motor, a buzzer, a camera, a microphone, and a sensor set. The communication unit can be based on wire or wireless telecommunication technology, e.g., RFID, NFC, Bluetooth, Zigbee, LTE, 3G, 4G, 5G, etc. The communication unit can be configured to receive data/information, e.g., an RFID reader, an NFC reader, a Bluetooth receiver, a bar code reader, or an OCR reader, and/or be configured to send out a signal, e.g., Bluetooth Beacon, RFID/NFC transmitter. The sensor set comprises at least one of a gyro sensor, an accelerometer, a temperature sensor and a photo sensor.

The housing 10 comprises an inner housing 102. The inner housing 102 comprises an inner wall 1021.

The medicament delivery device comprises a plunger rod operably connected to the stepping motor 13 such that the plunger rod is moved by the stepping motor into the medicament container to expel contained medicament. In a preferred example, the plunger rod is connected to the stepping motor via a lead screw. For example, the plunger rod is removably attached to the proximal end of the lead screw. The lead screw 11 extends along a longitudinal axis L between a proximal end and a distal end within the housing 10. In one example, the housing 10 extends along the longitudinal axis L between a proximal end and a distal end, as shown in FIGS. 1-2. Alternatively, the longitudinal axis of the lead screw is transverse to the axis that the housing extends along. The lead screw 11 comprises a thread 110 extending between the proximal end and the distal end. The lead screw 11 comprises a plunger rod 111 configured to be moved into the cassette 2 to expel the medicament contained within the cassette 2. In one example, the inner housing 102 comprises a channel portion configured to guide the movement of the lead screw 11. Alternatively, the lead screw is the plunger rod.

The processor is configured to perform the above-mentioned method in particular for the purpose of detecting the plunger rod situation, e.g., a position of the plunger rod and/or moving rate of the plunger rod. For example, the processor can determine a position of the plunger rod based on detecting the rotation of the stepping motor.

The processor is configured to pause, slow, or restore the rotation of the stepping motor when the processor determines the stepping motor is stalled. Furthermore, as the processor is configured to detect a position of the plunger rod based on the rotation of the stepping motor, the processor is preferably programmed to calibrate the detection when the motor stall is detected. For example, calculating the rotation angle based on the previous measured rotation angle and time period of the detected motor stall. Alternatively, or additionally, the driver unit comprises an encoder and/or an optical sensor, e.g., an optical navigator, such as a mouse sensor, electrically connected to the processor. In this example, the position of the plunger rod can be detected by the encoder and/or the optical sensor.

Furthermore, in another example, the distal end of the lead screw 12 comprises a surface 121a facing in a direction transverse to the longitudinal axis L. The surface 121a is adjacent to the inner wall 1021 of the housing 10 when the distal end of the lead screw 12 is moved along the longitudinal axis L. The distal end of the lead screw 12 comprises a non-circular cross-section perpendicular to the longitudinal axis and the inner wall 1021 of the housing 10 comprises a non-circular cross-section perpendicular to the longitudinal axis L such that the distal end of the lead screw 12 is rotationally fixed to the housing 10. The distal end of the lead screw comprises an outer surface having the non-circular cross-section perpendicular to the longitudinal axis L; and the inner wall of the housing comprises an inner surface having the non-circular cross-section perpendicular to the longitudinal axis. In one example, the entire outer surface of the distal end of the lead screw has the non-circular cross-section perpendicular to the longitudinal axis L, namely, the non-circular cross-section can be observed from the distal end of the distal end of the lead screw and the proximal end of the distal end of the lead screw. Alternatively, a part of the outer surface of the distal end of the lead screw has the non-circular cross-section perpendicular to the longitudinal axis L, namely, the non-circular cross-section can only be observed from either the distal end of the distal end of the lead screw or the proximal end of the distal end of the lead screw. Similarly, the inner wall of the housing can be arranged that the entire inner surface of the inner wall having the non-circular cross-section perpendicular to the longitudinal axis L; or a part of the entire inner surface of the inner wall having the non-circular cross-section perpendicular to the longitudinal axis L.

As it is common to have a small gap between components during manufacturing for dealing with the tolerance issue, meaning that as the injection molding process might not generate components that are all identical, the components that are designed to be in contact with one another all the time in a product may cause a significant waste for those components that cannot fit into one another; therefore, the term ‘adjacent’ means two objects are ‘nearby’ one another but not necessarily in contact to one another.

In one example, an engagement formed between the distal end of the lead screw 12 and the inner wall 1021 of the housing 10 is a ribs-ribs engagement or a ribs-grooves engagement. In one example, the distal end of the lead screw 12 comprises a groove 121 in a nut body 120, and the inner wall 1021 comprises a rib 1021a positioned within the groove 121. The rib 1021a extends in the direction of the longitudinal axis L. When the distal end of the lead screw 12 is moved along the longitudinal axis L, the groove 121 moves along the rib 1021a. Alternatively, the distal end of the lead screw comprises a polygonal cross-section perpendicular to the longitudinal axis and the inner wall of the housing comprises a polygonal cross-section perpendicular to the longitudinal axis L When the distal end of the lead screw is moved along the longitudinal axis L, the distal end of the lead screw moves along the inner wall. Therefore, the rotation between the housing and the distal end of the lead screw can be prevented.

In a preferred example, the distal end of the lead screw can provide a function to determine the dosage to be expelled. In one example, the inner wall comprises a radial protrusion extending towards the longitudinal axis L. The radial protrusion is configured to block the proximal movement of the distal end of the lead screw, thus to block the proximal movement of the lead screw. Therefore, only a certain amount of the medicament within the cassette of the medicament delivery device can be expelled. In another example, the inner wall comprises multiple radial protrusions that are axially and circumferentially offset relative to one another. In this example, the different deliverable dosages can be determined by using distal end of the lead screws of different shapes. For example, the lead screw is formed by a main section and a distal section. The distal section is removably attached to the main section to form a lead screw. In one example, the distal section is exchangeable. In one example, a set of distal section of the lead screws can be provided. Each of the distal section of the lead screws is formed with the cross-section shape as viewed along the longitudinal axis L being different from one another. In this example, the distal end of the lead screw is defined by one of the different distal sections that is configured to be blocked at an axial position that is different from other distal sections relative to the housing by at least one of the multiple protrusions of the inner wall. For example, one of the distal section of the lead screws comprises a triangle cross-section as viewed along the longitudinal axis L; another one of the distal section of the lead screws comprises a rectangular cross-section as viewed from the longitudinal axis L. In this example, at least one of the multiple protrusions of the inner wall of the housing is configured to block the rectangular distal section of the lead screw in a position; but the at least one of the multiple protrusions is configured to be spaced apart from the triangle distal section of the lead screw. Thus, the triangle distal end of the lead screw can be blocked in another position that is more proximal than the rectangular distal section of the lead screw.

It should be noted that, as the drive unit 1 can control the deliverable dosage by controlling the motor, using the distal end of the lead screw to provide the function of determining the dosage to be expelled is optional. In one example where the distal end of the lead screw provides the function of determining the dosage, the housing can be universal, and the moving distance of the lead screw can be limited by using different distal end of the lead screws. The hard stop of the lead screw can be used as a safety mechanism to limit the maximum derivable dose of the medicament to the patients. Thus, even if the electronic section 16 is programmed with incorrect medicament information, e.g., error programming by the user or the cassette 2 comprises a wrong tag, e.g., by mistake in the production line, the risk of overdose can be reduced.

The stepping motor 13 is rotatable around the lead screw 11 within the housing 10. The stepping motor 13 is engaged with the lead screw 11 via a threaded engagement. The stepping motor 13 is connected to the electronic section 16 such that the electronic section 16 controls the rotation of the stepping motor 13. When the stepping motor 13 rotates, the torque is transferred to the lead screw 11 via the thread engagement between the lead screw 11 and the stepping motor 13. In one example, the drive 13 is connected to the electronic section 16 via a wire 131. As the lead screw 11 is rotationally fixed to the housing 10, the torque from the stepping motor 13 is converted to an axial movement of the lead screw 11. In a preferred example, the stepping motor 13 is a motor electrically connected to the switch 163. As shown in FIG. 5, the motor comprises a rotor 130 and a channel 133 extending through the rotor 130. The lead screw 11 is partially positioned within the channel 133. In a preferred example, the longitudinal axis L is the central axis of the motor. In one example, the channel comprises a thread 134 extending around the channel. The thread is engaged with the thread of the lead screw. Alternatively, the drive unit comprises a connector 135 operably connected to the rotor 130 such that the connector 135 is configured to be rotated by the rotor. In this example, a channel extends through the connector and is aligned with the channel of the rotor 130 along the longitudinal axis L. The channel of the connector 135 comprises a thread 134 extending around the channel of the connector 135. The thread 134 is engaged with the thread of the lead screw 11. In this example, the connector 135 is attached to the rotor 130. In a preferred example, the connector 135 and the rotor 130 are made of different materials. As the thread 134 of the connector 135 is configured to engage with the lead screw 11, the connector 135 can be selected with a material that will create low friction. In one example, the rotor 130 is made of metal and the lead screw 11 is made of metal, then the connector 135 is preferably to be made of plastic.

The switch 163 is arranged within the housing The switch 163 is movable between an inactive position where the processor and the stepping motor are both inactive and an active position where the processor is activated. Optionally, the switch 163 is also configured to be used to turn on the stepping motor 13. Upon activation of the switch 163, the rotation of the stepping motor 13 around the lead screw 11 is configured to move the lead screw 11 proximally so that a stopper of a medicament container M in the cassette 2 is moved by the lead screw 11.

The switch can be manually activated by a user of the medicament delivery device. In one example, the switch is a button protruding from an outer surface of the housing. Alternatively, the housing comprises a touch panel, and the switch is an electronic switch that can be activated via the touch panel. Alternatively, the switch can be moved from the inactive position to the active position when the cassette 2 is attached to the housing of the drive unit 1.

In a preferred example, the stepping motor 13 is configured to reciprocate the movement of the lead screw 11. In one example where the stepping motor 13 is a motor, the motor can be programmed that it should rotate in an opposite direction once a raised resistance is detected (for example, by detecting the increased current). Alternatively, the stepping motor 13 is designed that will only deliver a pre-set dose such that once the pre-set dose is delivered, the motor will rotate in an opposite direction. Alternatively, the stepping motor 13 is configured to rotate in an opposite direction once the switch is deactivated.

Therefore, when the switch 163 is activated, the rotor 130 of the motor starts to rotate and thus causes the lead screw 11 to expel the medicament out of the cassette 2. When the medicament container M is empty, the movement of the lead screw 11 towards an exit of the medicament container M will be blocked by the medicament container M. Thus, an increased resistance can be detected and thus the motor can start to rotate in an opposite direction. For example, if the motor is rotated in a clockwise direction around the longitudinal axis L to cause the medicament to be expelled, when increased resistance is detected, the motor can rotate in a counterclockwise direction around the longitudinal axis L. Therefore, as the lead screw 11 is rotationally fixed to the housing 10, the lead screw 11 is moved distally.

In one example where the drive unit 1 comprises the second switch accommodated within the housing 10. The second switch is aligned with the distal end of the lead screw 12 in the direction of the longitudinal axis L. The second switch is positioned distally away from the distal end of the lead screw 12. The second switch is configured to be switched by the distal end of the lead screw 12 when the lead screw is moved distally relative to the stepping motor such that when the second switch is switched the motor is stopped. Therefore, at the end of the medicament delivery operation, the lead screw 11 is moved in the distal direction relative to the housing 10 by the motor as mentioned above, the motor keeps rotating until the second switch is switched off by the distal end of the lead screw 12. In a preferred example, when the second switch is switched by the distal end of the lead screw 12, the second switch is configured to switch off the entire electronic section 16. Alternatively, the second switch is configured to turn the electronic section 16 into the low power consumption mode.

In one example, the second switch is configured to be switched by being pushed in the distal direction relative to the housing by the distal end of the lead screw. Additionally, the second switch comprises a flipping arm. The second switch is configured to be switched when the flipping arm is pivoted by the distal end of the lead screw 12, as shown in FIG. 8. In this example, the second switch optionally comprises a flexible member, e.g., a spring or a flexible arm, configured to push the second switch towards the distal end of the lead screw 12 such that when the distal end of the lead screw 12 is moved apart from the second switch, the second switch is moved to its original position. Alternatively, the second switch is a reed switch, and the distal end of the lead screw comprises a magnet. Therefore, the distal end of the lead screw does not need to be physically in contact with the second switch to switch the second switch.

Furthermore, in one example where the drive unit 1 comprises the communication unit. In this example, the communication unit is configured to be activated when the switch 163 is activated. Another aspect of the invention that provides a medicament delivery device comprises the drive unit 1 and the cassette 2. The cassette 2 comprises a body 20 having the counter fixture 202 releasably attached to the fixture 101 of the housing 10 of the drive unit 1 and the delivery member cover 21.

The body 20 extends along an axis between a proximal end and a distal end. The delivery member cover 21 comprises a proximal portion 210 and a distal portion 211.

The delivery member cover 21 is telescopically relative to the proximal end of the body 20 between an extended position where the delivery member cover 21 protrudes from the proximal end of the body 20 in the direction of the axis L, and a retracted position.

1-15. (canceled)

16. A method for detecting a state of stall in a rotating stepping motor, the method comprising:
measuring the back EMF value of the rotating stepping motor;
calculating a first spread of the back EMF value, wherein the first spread of the back EMF value is defined by a difference between a highest back EMF value and a lowest back EMF value in a first fluctuation of the back EMF value when the stepping motor is rotating;
calculating a second spread of the back EMF value, wherein the second spread of the back EMF value is defined by a difference between a highest back EMF value and a lowest back EMF value in a second fluctuation of the back EMF value when the stepping motor is rotating;
comparing the second spread to the first spread; and
determining that the stepping motor is stalled when a difference between the second spread and the first spread is higher than a first predetermined value.

17. The method according to claim 16, wherein the step of measuring the back EMF value of the rotating stepping motor further comprises:
recording a back EMF value; and
starting to calculate the first spread and the second spread only when the recorded back EMF value is lower than a second predetermined value.

18. The method according to claim 17, wherein the second predetermined value is defined by:
obtaining the rotation speed of the stepping motor; and
determining the second predetermined value by searching data from a first predetermined table with the obtained rotation speed of the stepping motor.

19. The method according to claim 18, wherein the step of obtaining the rotation speed of the stepping motor comprises the step of:
measuring the rotation speed of the stepping motor.

20. The method according to claim 18, wherein the step of obtaining the rotation speed of the stepping motor comprises the step of:
receiving signal containing a predetermined rotation speed of the stepping motor.

21. The method according to claim 16, the method further comprising:
measuring the back EMF value of the rotating stepping motor;
comparing the measured value to a third predetermined value; and
determining a value of load of the stepping motor.

22. The method according to claim 21, wherein before the step of determining the value of load of the stepping motor, the method comprises the steps of:
obtaining the rotation speed of the stepping motor; and
determining the third predetermined value by searching data from a second predetermined table with the obtained rotation speed of the stepping motor.

23. A processor of a drive unit of a medicament delivery device, wherein the drive unit comprises a stepping motor, the processor being configured to perform the method according to claim 16.

24. A drive unit of a medicament delivery device comprising:
a stepping motor;
a plunger rod operably connected to the stepping motor; and
the processor according to claim 23, the processor being electrically connected to the stepping motor, wherein the plunger rod is configured to be driven by the stepping motor when the stepping motor is rotating.

25. The drive unit according to claim 24, wherein the processor is configured to pause, slow, or restore the rotation of the stepping motor when the processor determines the stepping motor is stalled.

26. The drive unit according to claim 24, wherein the processor is configured to determine a position of the plunger rod based on detecting the rotation of the stepping motor.

27. The drive unit according to claim 24, wherein the drive unit comprises a housing configured to receive a cassette of a medicament delivery device, the cassette being releasably attached to the housing, and wherein the plunger rod is configured to be moved into a medicament container within the cassette.

28. The drive unit according to claim 24, wherein the drive unit comprises a switch movable between an inactive position where the processor and the motor are both inactive and an active position wherein the processor is activated.

29. The drive unit according to claim 28, wherein the switch is configured to be moved from the inactive position to the active position when the cassette is attached to the housing.

30. A medicament delivery device comprising the drive unit according to any claim 24, wherein the medicament delivery device is an injection device.
